(19) United States
(12) Patent Application Publication
(10) Pub. No.: US 2018/0008422 A1
(43) Pub. Date: Jan. 11, 2018

(54) KNEE ARTHROPLASTY SYSTEM

(71) Applicant: Biomet Manufacturing, LLC, Warsaw, IN (US)

(72) Inventors: Robert Metzger, Wakarusa, IN (US); Brian M. May, Warsaw, IN (US); Rusell Lloyd, Swindon Wiltshire (GB)

(21) Appl. No.: 15/636,152

(22) Filed: Jun. 28, 2017

Related U.S. Application Data
(60) Provisional application No. 62/359,863, filed on Jul. 8, 2016.

(51) Int. Cl. A61F 2/38; A61F 2/30

(52) U.S. Cl. CPC A61F 2/389; A61F 2/3859; A61F 2/30771; A61F 2002/30607; A61F 2002/30382; A61F 2002/30934; A61F 2002/3895; A61F 2002/30515

(57) ABSTRACT

An arthroplasty system including a unicondylar tibial tray implant, a total tibial tray implant, and a tibial bearing configured to individually couple with each one of the unicondylar tibial tray implant and the total tibial tray implant.

### CLAIM OF PRIORITY

This application claims the benefit of U.S. Provisional Patent Application Ser. No. 62/359,863, filed on Jul. 8, 2016, the benefit of priority of which is claimed hereby, and which is incorporated by reference herein in its entirety.

### FIELD

The present disclosure relates to a knee arthroplasty system.

### BACKGROUND

This section provides background information related to the present disclosure, which is not necessarily prior art.

Knee arthroplasty typically calls for implanting either a partial (unicondylar) or a total arthroplasty device, depending on the condition of the patient's knee (i.e., the extent to which the knee has been damaged due to wear or trauma). Although existing partial and total knee devices and systems are often marketed under a common brand name, they do not share design elements. In other words, unicondylar and total arthroplasty devices and systems are designed independently from one another based on different anatomic data. As a result, the kinematics between the two systems is different, as is sizing rational and surgical technique, for example. This leads to inefficient use of implant inventory and instrumentation. There is thus a need for improved knee arthroplasty systems having enhanced efficiencies. For example, knee arthroplasty systems that provide a consistent approach to treating osteoarthritis, such as by providing multiple treatment options along one continuum, from unicondylar to bi-compartmental to tri-compartmental (total) replacement, would therefore be desirable. The present teachings provide for such advantages, as well as numerous others.

### SUMMARY

This section provides a general summary of the disclosure, and is not a comprehensive disclosure of its full scope or all of its features.

The present teachings provide for an arthroplasty system including a unicondylar tibial tray implant, a total tibial tray implant, and a tibial bearing configured to individually couple with each one of the unicondylar tibial tray implant and the total tibial tray implant.

The present teachings further provide for an arthroplasty system including a unicondylar tibial tray implant having a unicondylar bearing engaging surface, and a total tibial tray implant having a medial bearing engaging surface and a lateral bearing engaging surface. The unicondylar bearing engaging surface is substantially similar to the medial or the lateral bearing engaging surfaces in size and shape.

The present teachings also provide for a unicondylar tibial tray implant, a total tibial tray implant, a tibial bearing, a unicondylar femoral implant, and a total femoral implant. The unicondylar tibial tray implant includes a unicondylar bearing engaging surface. The total tibial tray implant includes a medial bearing engaging surface, a lateral bearing engaging surface, and an intercondylar portion between the medial bearing engaging surface and the lateral bearing engaging surface. The unicondylar bearing engaging surface is substantially similar to the medial or the lateral bearing engaging surfaces in size and shape. The tibial bearing is configured to couple with the unicondylar tibial tray implant at the unicondylar bearing engaging surface, and configured to couple with the total tibial tray implant at one of the medial bearing engaging surface and the lateral bearing engaging surface. Each one of the unicondylar femoral implant and the total femoral implant are configured to articulate with the tibial bearing. Each one of the unicondylar tibial try implant, the total tibial tray implant, the tibial bearing, the unicondylar femoral implant, and the total femoral implant are sized and shaped based on the same anatomic data.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1A is an exploded view of a unicondylar tibial implant assembly according to the present teachings;

FIG. 1B is a perspective view of a tibial bearing of the unicondylar tibial implant assembly of FIG. 1A;

FIG. 1C is a planar, superior view of the tibial bearing;

FIG. 1D is a planar, superior view of a unicondylar tibial tray implant of the unicondylar tibial implant assembly of FIG. 1A;

FIG. 2A is a perspective view of a total tibial tray implant assembly according to the present teachings;

FIG. 2B is a planar view of the total tibial tray implant assembly of FIG. 2A without bearings coupled thereto;

FIG. 3A is an exploded view of another total tibial tray implant assembly according to the present teachings;

FIG. 3B is a superior, planar view of the total tibial tray implant assembly of FIG. 3A without bearings coupled thereto;

FIG. 4 is a perspective view of the total tibial tray implant assembly of FIG. 3A and a total bearing;

FIG. 5A is a perspective view of a unicondylar femoral component according to the present teachings;

FIG. 5B is a planar view of the unicondylar femoral component according to the present teachings;

FIG. 5C is a side view of the unicondylar femoral component of FIG. 5A;

FIG. 6A is a perspective view of a total femoral component according to the present teachings;

FIG. 6B is a side view of the total femoral component of FIG. 6A; and

FIG. 6C illustrates medial and lateral articulating surfaces of the total femoral component of FIG. 6A.

Corresponding reference numerals indicate corresponding parts throughout the several views of the drawings.

### DETAILED DESCRIPTION

Example embodiments will now be described more fully with reference to the accompanying drawings.

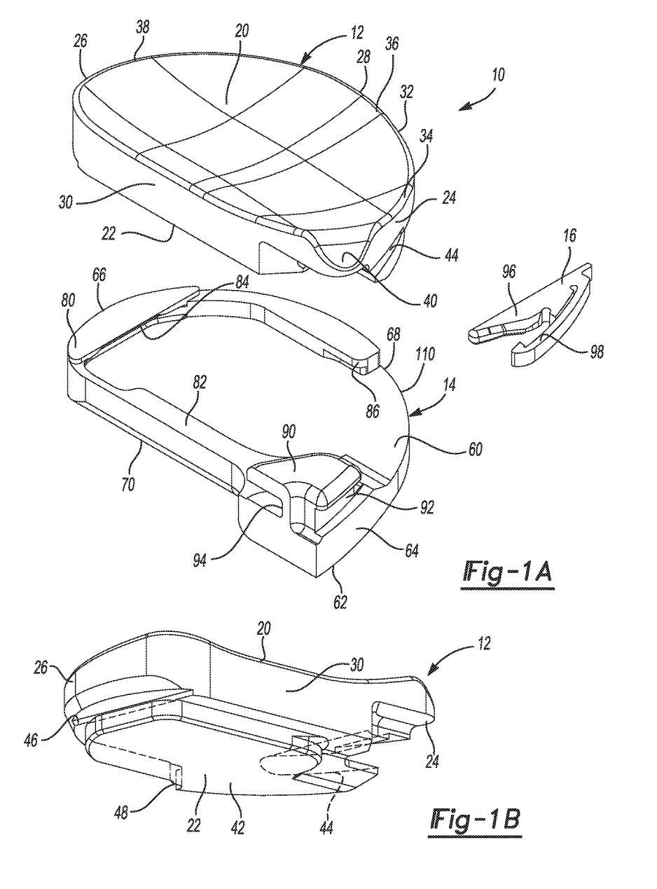

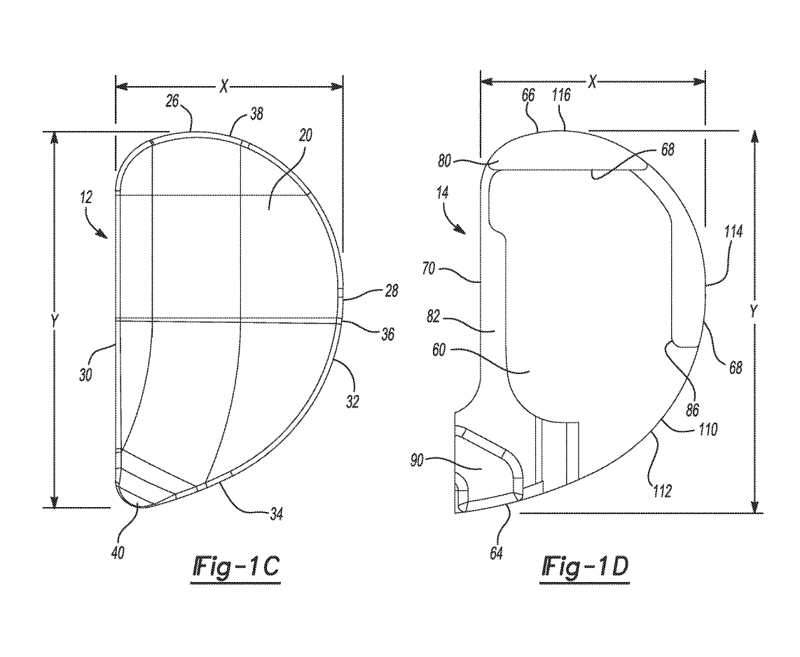

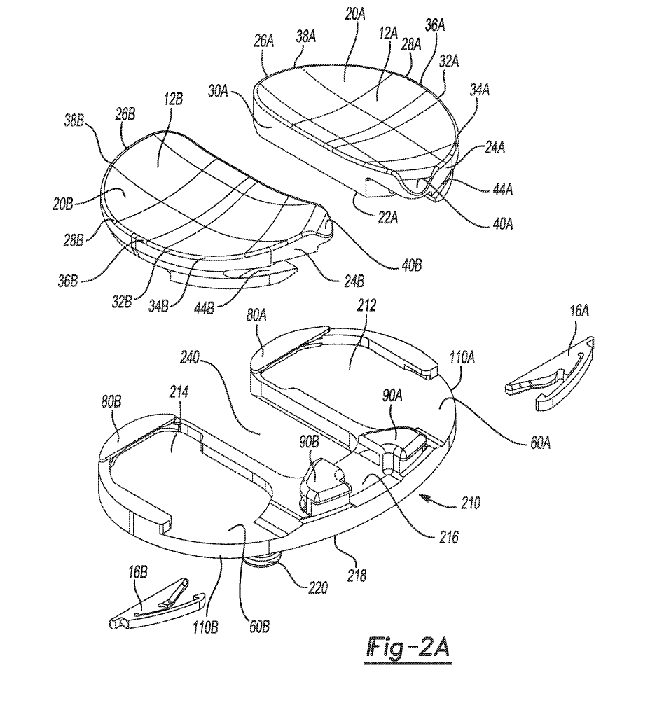

With initial reference to FIG. 1A, a unicondylar tibial implant assembly according to the present teachings is generally illustrated at reference numeral 10. The unicondylar tibial implant assembly 10 is generally described herein as a lateral assembly, for exemplary purposes only. The present teachings further provide for a medial unicondylar tibial implant assembly, which one skilled in the art will recognize can be a mirror image of, or a substantial mirror image of, the lateral unicondylar tibial implant assembly 10. The description herein of the lateral unicondylar tibial implant assembly 10 is thus sufficient to also describe a medial unicondylar tibial implant assembly.

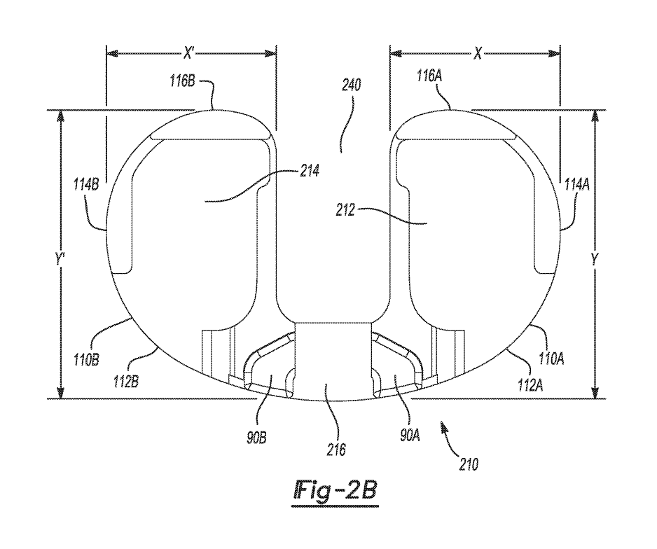

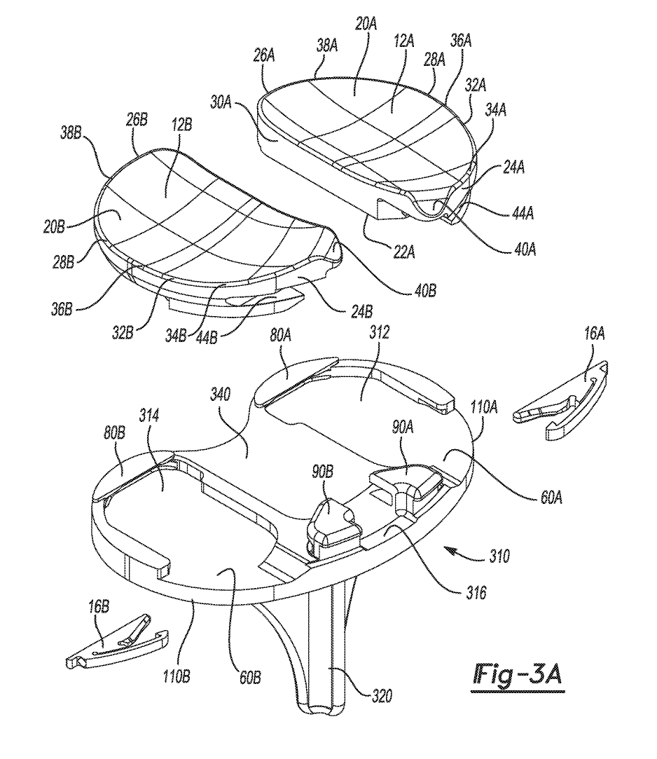

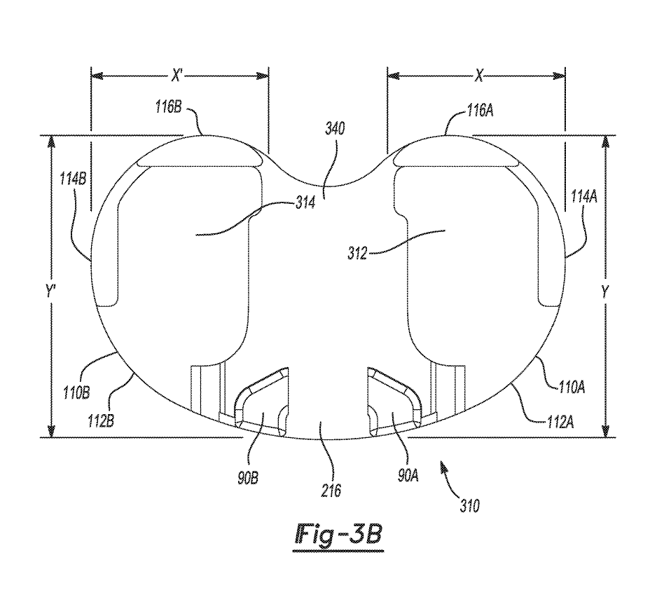

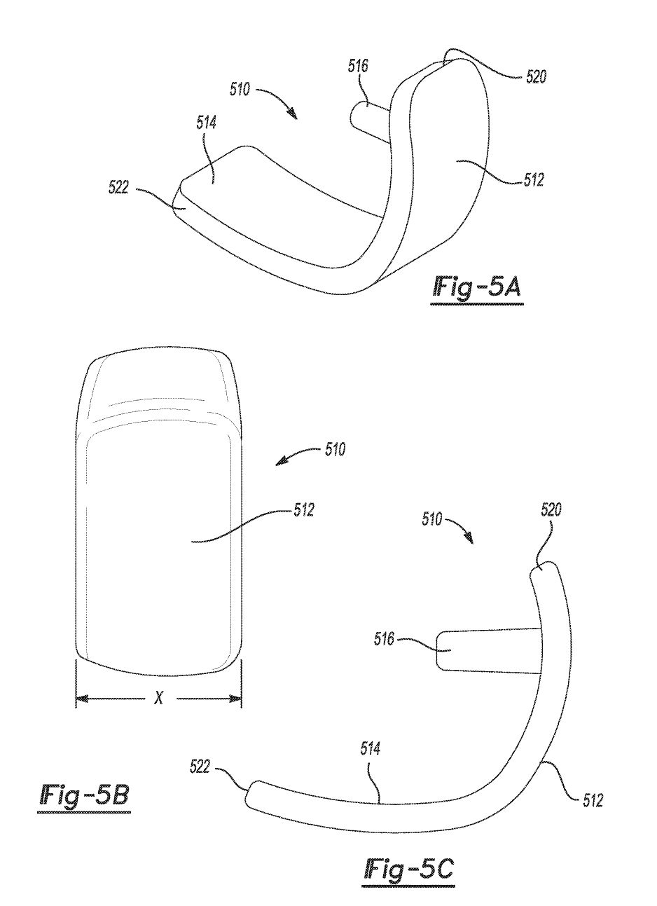

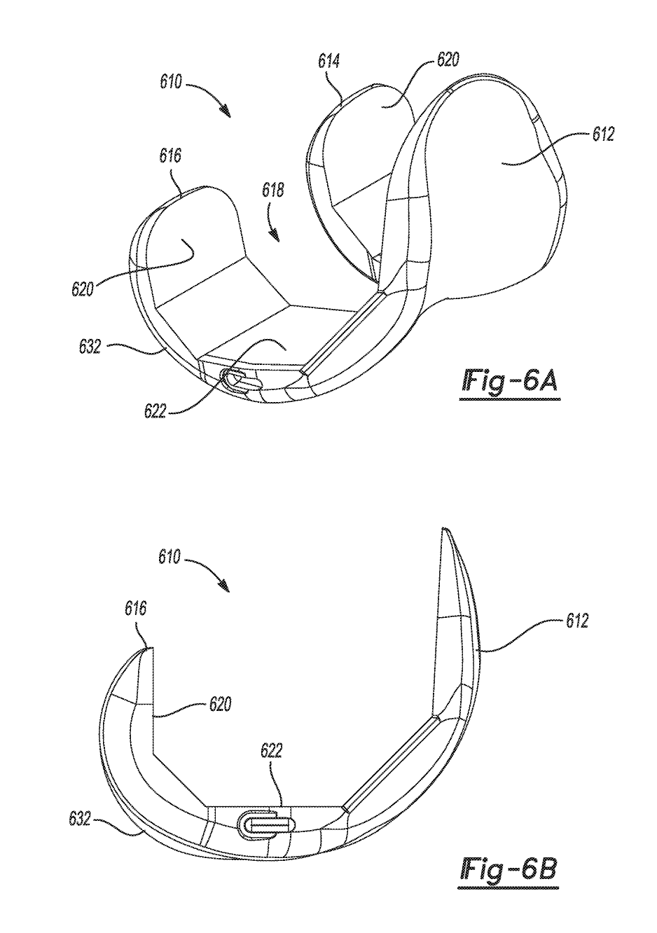

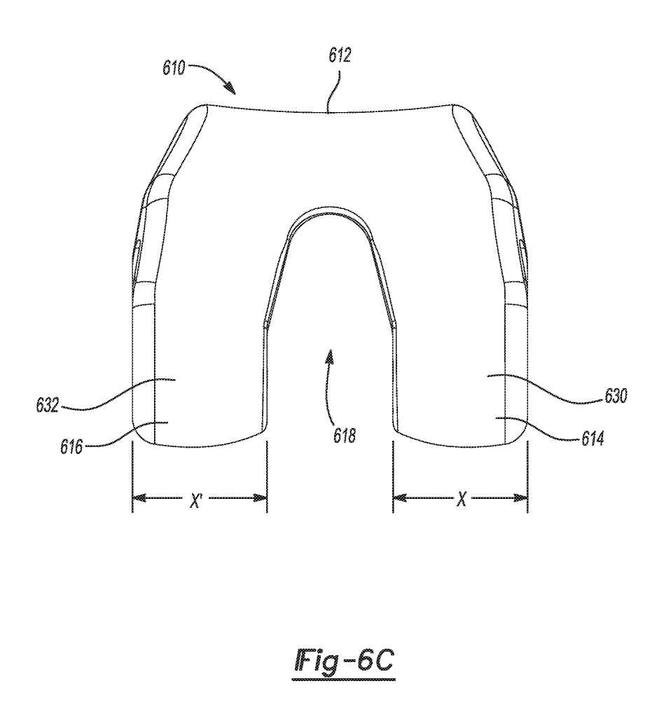

The lateral unicondylar tibial implant assembly 10 generally includes a tibial bearing 12, a unicondylar tibial tray implant 14, and a locking member or locking bar 16. The tibial bearing 12 generally includes a unicondylar articulation surface 20, which is opposite to a unicondylar tray engaging surface 22. The articulation surface 20 is configured to articulate with either a unicondylar femoral component 510 (FIG. 5A), or a total femoral component 610 (FIG. 6A), as described further herein. The unicondylar tray engaging surface 22 is configured to couple to the unicondylar tibial tray implant 14, or a total tibial tray implant (FIGS. 2A, 2B, 3A, 3B), as explained herein.

With continued reference to FIG. 1A and additional reference to FIGS. 1B and 1C, the tibial bearing 12 further includes an anterior end 24, a posterior end 26, a lateral side 28, and a medial side 30. A curved outer bearing surface 32 extends about a substantial portion of the tibial bearing 12, such as from the anterior end 24, across the lateral side 28, and to or slightly beyond the posterior end 26. Thus the curved outer bearing surface 32 generally includes an anterior portion 34 at the anterior end 24, a lateral portion 36 at the lateral side 28, and a posterior portion 38 at the posterior end 26. The tibial bearing 12 further includes an angled or flap portion 40, generally between the anterior end 24 and the medial side 30.

With reference to FIG. 1B, for example, the tibial bearing 12 includes a coupling system or portion 42. The coupling portion 42 can be configured in any suitable manner in order to couple the tibial bearing 12 to the unicondylar tibial tray implant 14, as well as to any suitable total tibial tray implant, such as the total tibial tray implants illustrated in FIGS. 2A, 2B, 3A, and 3B, and as further described herein. For example, the coupling portion 42 can include an anterior coupling recess 44, a posterior coupling recess 46, and a lateral coupling flange 48. The anterior coupling recess 44 is generally at the anterior end 24 of the tibial bearing 12, and is configured to receive the locking member 16 in order to secure the tibial bearing 12 to the unicondylar tibial tray implant 14. The anterior coupling recess 44 generally extends in the lateral to medial direction, such as from the lateral side 28 to the medial side 30. The posterior coupling recess 46 is at the posterior end 26 and is generally a recess or slot extending in the lateral to medial direction, such as from the lateral side 28 to the medial side 30. The lateral coupling flange 48 is at the lateral side 28 of the tibial bearing 12.

With reference to FIGS. 1A and 1D for example, the unicondylar tibial tray implant 14 generally includes a unicondylar bearing engaging surface 60 configured to mate with the tibial bearing 12, and a unicondylar bone engaging surface 62, which is opposite to the unicondylar bearing engaging surface 60. The unicondylar bone engaging surface 62 is configured to be coupled to a tibia bone, and thus may include any suitable retention member to facilitate retention of the unicondylar tibial tray implant 14 at the bone, such as a retention post, retention fin, or retention rail.

The unicondylar tibial tray implant 14 further includes an anterior end 64, which is opposite to a posterior end 66. Between the anterior end 64 and the posterior end 66 is a lateral side 68 and a medial side 70. The unicondylar tibial tray implant 14 includes a coupling system or portion 80, which can be any suitable coupling system or portion configured to couple the tibial bearing 12 thereto, or any other suitable unicondylar or total tibial bearing.

The coupling portion 80 in the example illustrated generally includes a raised portion 82, which generally extends across the medial side 70, across the posterior end 66, and across at least a portion of the lateral side 68. The raised portion 82 generally extends from the unicondylar bearing engaging surface 60, and defines a posterior coupling flange 84 at the posterior end 66. The posterior coupling flange 84 is configured to cooperate with the posterior coupling recess 46 of the lateral tibial bearing 12. At the lateral side 68, the raised portion 82 defines a lateral coupling flange 86, which is configured to cooperate with the lateral coupling flange 48 of the tibial bearing 12. The posterior coupling flange 84 and the lateral coupling flange 86 thus respectively retain the posterior end 26 and the lateral side 28 of the lateral tibial bearing 12 to the unicondylar tibial tray implant 14.

The coupling portion 80 of the unicondylar tibial tray implant 14 further includes an anterior locking post 90 proximate to the anterior end 64 and extending in the superior direction, such as away from the unicondylar bearing engaging surface 60. The anterior locking post 90 generally defines an outer recess 92 and an inner recess 94. The anterior locking post 90 is configured to couple with the locking bar 16 in order to secure the anterior end 24 of the tibial bearing 12 to the anterior end 64 of the unicondylar tibial tray implant 14. Specifically, when the tibial bearing 12 is seated on the unicondylar bearing engaging surface 60, the locking bar 16 is inserted through the anterior coupling recess 44 and into cooperation with the anterior locking post 90. A bearing engaging portion 96 of the lateral locking bar 16 extends through the anterior coupling recess 44 and into cooperation with the anterior locking post 90 at the inner recess 94 thereof. A locking post engaging portion or clip 98 of the lateral locking member 16 contacts and engages the outer recess 92 of the anterior locking post 90 in order to retain the locking bar 16 in cooperation with the anterior locking post 90.

With continued reference to FIG. 1A, and additional reference to 1D, the lateral unicondylar tibial tray implant 14 further includes a curved outer tray surface 110, which extends from the anterior end 64, across the lateral side 68, and slightly beyond the posterior end 66 to the medial side 70. The curved outer tray surface 110 generally includes, with reference to FIG. 1D, an anterior portion 112, a lateral portion 114, and a posterior portion 116. The curved outer tray surface 110 is generally similar to the curved outer bearing surface 32 of the tibial bearing 12. Thus, both the curved outer tray surface 110 and the curved outer bearing surface 32 have the same or substantially similar size, shape, and curve radius at each of the anterior portions 112/34, the lateral portions 114/36, and the posterior portions 116/38. As illustrated in FIGS. 1C and 1D, the lateral tibial bearing 12 and the unicondylar tibial tray implant 14 also have substantially similar or the same maximum anterior to posterior dimensions “Y,” and substantially similar or the same maximum medial to lateral dimensions “X”.

With additional reference to FIGS. 2A and 2B, a total tibial tray implant assembly according to the present teachings is illustrated at reference numeral 210. The total tibial tray implant assembly 210 is configured to couple with the lateral tibial bearing 12, illustrated at 12A in FIGS. 2A and 3A, as well as the medial version thereof, which is illustrated in FIGS. 2A and 3A at reference numeral 12B. The suffix's “A” and “B” are used in FIGS. 2A and 3A to distinguish the lateral and medial tibial bearings 12A and 12B respectively. As explained above, the medial tibial bearing 12B is a mirror image of, or a substantial a mirror image of, the lateral tibial bearing 12A. Therefore, the description above of the lateral tibial bearing 12A also applies to the medial tibial bearing 12B.

The total tibial tray implant assembly 210 generally includes a lateral tray 212, a medial tray 214, and an intercondylar bridge portion 216, which is between and connects the lateral and medial trays 212 and 214. Extending from a bone engaging surface 218 can be any suitable retention member configured to facilitate retention of the total tibial tray implant assembly 210 to a tibia bone, such as one or more retention posts 220. The total tibial tray implant assembly 210 can be any suitable total tibial tray implant, such as a total tibial tray implant configured to accommodate a natural ACL in an intercondylar slot 240 defined between the lateral tray 212 and the medial tray 214.

The lateral tray 212 is substantially similar to the lateral unicondylar tibial tray implant 14. Therefore, features of the lateral tray 212 that are similar to, or the same as, features of the lateral unicondylar tibial tray implant 14 are designated with the same reference numerals, but include the suffix “A,” and the description of such features in conjunction with the unicondylar tibial tray implant 14 also applies to the lateral tray 212. The lateral tray 212 is illustrated as including the same coupling system or portion 80A as the lateral unicondylar tibial tray implant 14. However, the lateral tray 212 can include any other suitable coupling device, portion, or features to secure the lateral tibial bearing 12A thereto.

The lateral tray 212 includes a lateral curved outer tray surface 110A, which is substantially similar to, or the same as, the curved outer tray surface 110 of the lateral unicondylar tibial tray implant 14. Therefore, and with additional reference to FIG. 2B for example, the anterior portion 112A, the lateral portion 114A, and the posterior portion 116A of the total tibial tray implant assembly 210 are substantially similar to, or the same as, the anterior, lateral, and posterior portions 112, 114, and 116 of the curved outer tray surface 110 of the unicondylar tibial tray implant 14 in terms of at least size, shape, and curve radius. The anterior, lateral, and posterior portions 34A, 36A, and 38A of the curved outer bearing surface 32A of the tibial bearing 12A are also substantially similar to, or the same as, the anterior, lateral, and posterior portions 112A, 114A, and 116A of the curved outer tray surface 110A in terms of at least size, shape, and curve radius. This is because, for example, the unicondylar tibial tray implant 14 and the lateral tray 212 are based on the same anatomical data. The same tibial bearing 12/12A is thus able to mate with both the unicondylar tibial tray implant 14 and the lateral tray 212 of the total tibial tray implant assembly 210. This reduces the need for a specific set of bearings configured to couple to the unicondylar tibial tray implant 14, as well as an additional specific set of bearings configured to couple with the lateral tray 212, thereby reducing manufacturing costs, design costs, and storage costs.

The medial tray 214 is a mirror image of, or substantially a mirror image of, the lateral unicondylar tibial tray implant 14 and the lateral tray 212. Therefore similar features are designated with the same reference numerals, but include the suffix “B.” The description of the similar or like features set forth above in the description of the lateral unicondylar tibial tray implant 14 and the lateral tray 212 also applies to the medial tray 214. The medial tibial bearing 12B is thus configured to couple with both the unicondylar medial version (not specifically illustrated) of the lateral unicondylar tibial tray implant 14, as well as the medial tray 214. The medial tibial bearing 12B can be coupled to the medial tray 214 with a medial locking member 16B, which is substantially similar to the lateral locking member 16A.

The medial tray 214 includes a curved outer tray surface 110B, which is substantially similar to a curved outer tray surface of the medial version of the unicondylar lateral tibial tray implant 14. The curved outer tray surface 110B is thus a mirror image of, or substantially a mirror image of, both the curved outer tray surface 110 of the unicondylar tibial tray implant 14, and the curved outer tray surface 110A of the lateral tray 212. The medial curved outer tray surface 110B includes, at illustrated in FIG. 2B, an anterior portion 112B, a medial portion 114B, and a posterior portion 116B. The anterior, medial, and posterior portions 112B, 114B, and 116B are the same as, or substantially similar to, anterior, medial, and posterior portions 34B, 36B, and 38B of the curved outer bearing surface 32B of the medial tibial bearing 12B in terms of at least size, shape, and curve radius. The anterior, medial, and posterior portions 112B, 114B, and 116B are also the same as, or substantially similar to, anterior, medial, and posterior portions of the medial version of the lateral unicondylar tibial tray implant 14. The medial unicondylar tibial tray implant, the medial tibial bearing 12B, and the medial tray 214 are based on the same anatomical data.

As illustrated in FIG. 2B, the lateral tray 212 has a medial to lateral width X that is substantially similar to, or the same as, a medial to lateral width X of both the lateral unicondylar tibial tray implant 14 (FIG. 1D) and the lateral unicondylar tibial bearing 12 (FIG. 1C). The lateral tray 212 also has an anterior to posterior length Y that is substantially similar to, or the same as, an anterior to posterior length Y of both the lateral unicondylar tibial tray implant 14 (FIG. 1D) and the lateral unicondylar tibial bearing 12 (FIG. 1C). The medial tray 214 has a medial to lateral width X′ that is substantially similar to, or the same as, a medial to lateral width of the medial tibial bearing 12B and the lateral version of the unicondylar tibial tray implant 14. The medial tray 214 also has an anterior to posterior length Y′ that is substantially similar to, or the same as, an anterior to posterior length of the tibial bearing 12B, and an anterior to posterior length of the medial version of the lateral unicondylar tibial tray implant 14. This again is because the medial tray 214, the tibial bearing 12B, and the medial version of the lateral unicondylar tibial tray implant 14 are based on the same anatomical data.

Although the total tibial tray implant assembly 210 is illustrated as including coupling systems or portions 80A and 80B, which are substantially similar to the coupling system or portion 80 of the lateral unicondylar tibial tray implant 14, the total tibial tray implant assembly 210 can include any other suitable bearing coupling system or portion suitable for retaining the lateral and medial tibial bearings 12A and 12B thereto that is compatible with the coupling system or portion 42 of the bearings 12A and 12B, or any other coupling system or portion that the bearings 12A and 12B may include.

With reference to FIG. 3A and FIG. 3B, an additional total tibial tray implant assembly according to the present teachings is illustrated at reference numeral 310. The total tibial tray implant assembly 310 generally includes a lateral tray 312, a medial tray 314, an intercondylar bridge portion 316, and a retention post 320. Unlike the total tibial tray implant assembly 210, the total tibial tray implant assembly 310 includes an intercondylar portion 340 in place of the intercondylar slot 240. Thus the total tibial tray implant assembly 310 is configured for use in an arthroplasty in which the ACL is not present.

The lateral tray 312 is substantially similar to both the lateral tray 212 and the lateral unicondylar tibial tray implant 14, and is based on the same anatomic data. Therefore, common features are illustrated in FIG. 3A using the same reference numerals with the suffix “A,” and the description of the lateral unicondylar tibial tray implant 14 and the lateral tray 212 also applies to the lateral tray 312. Similarly, the medial tray 314 is the same as, or substantially the same as, the medial tray 214 and the medial version of the lateral unicondylar tibial tray implant 14. Therefore, the features of the medial tray 314 that are common to both the medial tray 214 and the medial version of the lateral unicondylar tibial tray implant 14 are illustrated with the same reference numerals along with the suffix “B,” and the description of the medial tray 214 also applies to the medial tray 314. The same lateral and medial bearings 12A and 12B can thus couple with both the total tibial tray implant assembly 210 and the total tibial tray implant assembly 310, as well as with the lateral unicondylar tibial tray implant 14 and the medial version thereof.

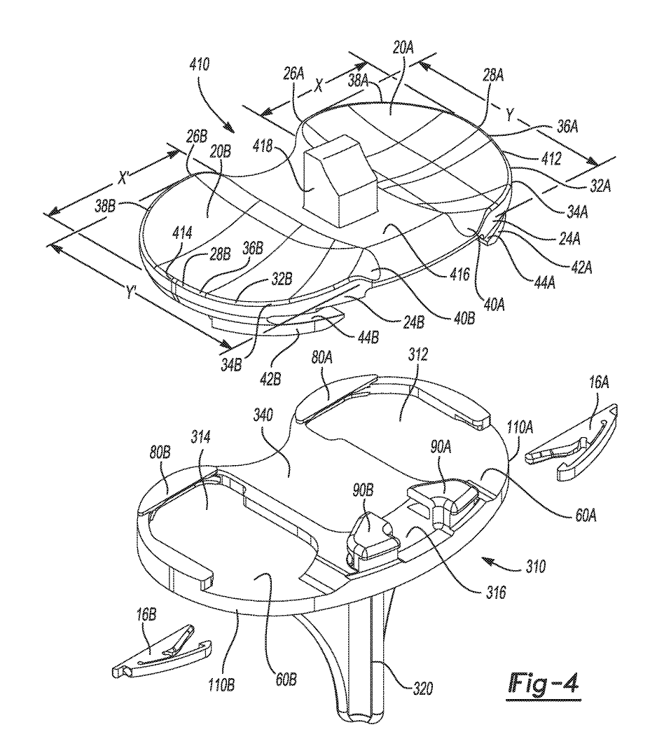

With reference to FIG. 4, the total tibial tray implant assembly 310 can couple with the lateral and medial tibial bearings 12A and 12B, as well as with a total bearing 410, which includes a lateral tibial bearing 412 and a medial tibial bearing 414 connected by a center portion 416. Extending superiorly from the center portion 416 is a center guidepost 418, which can cooperate with any suitable total femoral component in order to provide enhanced constraint between the total tibial tray implant assembly 310 and the femoral component. The lateral tibial bearing 412 is substantially similar to, or the same as, the lateral unicondylar tibial bearing 12/12A of the unicondylar tibial implant assembly 10, and thus similar features are illustrated with like reference numerals, and the description of the similar features set forth above with respect to the lateral unicondylar tibial bearing 12/12A also applies to the lateral tibial bearing 412. Likewise, the medial tibial bearing 414 is substantially similar to the medial unicondylar tibial bearing 12B, and thus like features are illustrated using the same reference numerals. The lateral tibial bearing 412 and the medial tibial bearing 414 are thus based on the same anatomic data that the unicondylar lateral and medial tibial bearings 12A and 12B are based on.

The total tibial tray implant assembly 310 can include the same coupling system or portions 80A and 80B described above, or any other suitable coupling portions or system configured to secure the total bearing 410 at the lateral and medial trays 312 and 314. Likewise, the total bearing 410 can include any suitable coupling portion or system to secure the total bearing 410 at the lateral and medial trays 312 and 314. As illustrated in FIG. 4, the total bearing includes the coupling system or portions 42A and 42B of the lateral and medial unicondylar tibial bearings 12A and 12B, and the total tibial tray implant assembly 310 includes the coupling system or portions 80A and 80B, as well as the locking bars 16A and 16B.

With reference to FIGS. 5A, 5B, and 5C, a unicondylar femoral component according to the present teachings is generally illustrated at reference numeral 510. The unicondylar femoral component 510 generally includes an articulation surface 512 and a bone engaging surface 514 opposite thereto. Extending from the bone engaging surface 514 is a retention post 516, which can be any suitable retention member configured to cooperate with a femoral bone in order to facilitate coupling of the unicondylar femoral component 510 to the femoral bone. The unicondylar femoral component 510 further includes a first end 520 and a second end 522 opposite thereto. The unicondylar femoral component 510 can be configured as either a lateral unicondylar femoral component or a medial unicondylar femoral component, as will be appreciated by one skilled in the art.

With reference to FIG. 5B, the articulation surface 512 includes a medial to lateral width “X.” The articulation surface 512 is sized and shaped to permit articulation with any suitable unicondylar or total tibial bearing. For example, when the unicondylar femoral component 510 is configured as a lateral component, the articulation surface 512 is configured to articulate with either the lateral unicondylar tibial bearing 12/12A or the lateral tibial bearing 412 of the total bearing 410, or any other suitable tibial bearing. When the unicondylar femoral component 510 is configured as a medial component, the articulation surface 512 can articulate with the medial unicondylar tibial bearing 12B or the medial tibial bearing 414 of the total bearing 410. When the unicondylar femoral component 510 is configured to be interchangeable between a medial and a lateral component, the unicondylar femoral component 510 can articulate with any of the lateral unicondylar tibial bearing 12/12A, the medial unicondylar tibial bearing 12B, the lateral articulation surface 20A of the total bearing 410, and the medial articulation surface 20B of the total bearing 410. Thus, the unicondylar femoral component 510 can be based on the same anatomic data as the medial and lateral unicondylar tibial bearings 12/12A/12B and the total bearing 410.

With reference to FIGS. 6A, 6B, and 6C, a total femoral component according to the present teachings is generally illustrated at reference numeral 610. The total femoral component 610 generally includes a patellar track portion 612, a lateral femoral condyle 614, and a medial femoral condyle 616. An opening/passage 618 is defined between the lateral femoral condyle 614 and the medial femoral condyle 616 in order to accommodate, for example, an ACL. The total femoral component 610 further includes a lateral bone engaging surface 620 and a medial bone engaging surface 622.

Opposite to the lateral bone engaging surface 620 is a lateral articulation surface 630. Opposite to the medial bone engaging surface 622 is a medial articulation surface 632. With particular reference to FIG. 6C, the lateral articulation surface 630 includes a medial to lateral width X, and the medial articulation surface 632 includes a medial to lateral width X′. The lateral articulation surface 630 is sized and shaped to permit articulation with the lateral tibial bearing 12/12A, as well as the lateral tibial bearing 412 of the total bearing 410. Similarly, the medial articulation surface 632 is configured with any suitable size and shape to articulate with the medial tibial bearing 12B, as well as the medial tibial bearing 414 of the total bearing 410.

When the unicondylar femoral component 510 is a lateral component, the medial to lateral width X of the articulation surface 512 can be the same as, or substantially similar to, the medial to lateral width X of the lateral articulation surface 630. When the unicondylar femoral component 510 is configured as a medial component, the articulation surface 512 can have a medial to lateral width X that is the same as, or substantially similar to, the medial to lateral width X′ of the medial articulation surface 632 of the total femoral component 610.

The present teachings thus provide for the lateral unicondylar tibial bearing 12/12A, the medial unicondylar tibial bearing 12B, the lateral articulation surface 20A, the medial articulation surface 20B, the lateral tray 212, the medial tray 214, the unicondylar femoral component 510, and the total femoral component 610 to be based on the same anatomic data, which is often complied based on measurements of a plurality of different knee joints from a statistically suitable segment of the population. This means that the same anatomic data and kinematics, such as femoral and tibial sizing, femoral and tibial orientation, inferior-superior translation, internal-external rotation, varus-valgus rotation, anterior-posterior translation, flexion-extension rotation, and medial-lateral translation, is used to design each one of the lateral unicondylar tibial bearing 12/12A, the medial unicondylar tibial bearing 12B, the lateral articulation surface 20A, the medial articulation surface 20B, the lateral tray 212, the medial tray 214, the unicondylar femoral component 510, and the total femoral component 610. Any particular anatomic data and kinematics can be used, as long as it is the same.

The present teachings provide for common sizing and design rationale across each tibial and femoral component. Any suitable number of sizes of the tibial and femoral components can be provided to fit a suitably wide segment of the population. For example, ten different sizes of each of the following components can be provided: lateral tibial bearings 12/12A; medial tibial bearings 12B; lateral unicondylar tibial tray implants 14; medial unicondylar tibial tray implants (not specifically illustrated); total bearings 410; total tibial trays 210; total tibial trays 310; unicondylar femoral components 510; and total femoral components 610. All of these femoral and tibial components are based on the same anatomic and kinematic data as described above. Therefore, the bearings 12/12A/12B of a particular size can couple with similarly sized tibial tray implants 14 and total tibial trays 210 and 310. The total bearing 410 of a particular size can couple with similarly sized total tibial trays 210 and 310. The unicondylar femoral component 510 and the total femoral component 610 can articulate with bearings 12/12A/12B and 410 of the same size. For example, the largest lateral tibial bearing 12A is configured to couple with the largest lateral unicondylar tibial tray implant 14, the largest lateral tray 212, and the largest lateral tray 312. The largest tibial bearing 12A also has a curved outer bearing surface 32 that is the same size and shape as the curved outer tray surface 110 of the largest lateral unicondylar tibial tray implant 14, and the same size and shape as the curved outer tray surface 11 OA of the largest total tibial trays 210 and 310. The largest unicondylar femoral component 510 is configured to articulate with the largest tibial bearing 12/12A/12B, as well as the largest total bearing 410. The largest total femoral component 610 is configured to articulate with the largest tibial bearing 12/12A/12B and the largest total bearing 410.

The total femoral components 610 have anterior geometry at the patellar track portion 612 that is consistent with a patellofemoral replacement device. All total femoral components 610 thus allow for use of the same patellar implant, thereby further reducing inventory and manufacturing costs. All femoral components can have the same sagittal profiles, which provides the same kinematic patterns, regardless of whether the ACL is retained or artificially replaced.

The common sizing rational of the present teachings provides numerous advantageous, such as inventory reduction and a seamless between implants during surgery and/or revision procedures, such as between unicondylar implants, ACL preserving, and ACL replacing implants. The present teachings thus advantageously provide for a consistent approach to treating osteoarthritis, which provides multiple treatment options along one continuum from unicondylar, to bicompartmental, to total knee replacement.

The present teachings also provide for multiple modular articulation surfaces for providing varying levels of constraint. For example, in addition to the total bearing 410 including the center guidepost 418 to provide enhanced constraint with the femoral component 610. Another total bearing 410 can be provided without the guidepost 418 to provide less constraint. On the femoral side, additional total femoral components 610 can be provided including an intercondylar portion or box, and/or a stem, which can provide further support for a fully constrained total femoral component.

The commonalities described above between the lateral unicondylar tibial bearing 12/12A, the medial unicondylar tibial bearing 12B, the lateral articulation surface 20A, the medial articulation surface 20B, the lateral tray 212, the medial tray 214, the unicondylar femoral component 510, and the total femoral component 610 also permit use of common instrumentation to prepare the femur and tibia regardless of whether a unicondylar or total knee arthroplasty is being performed. For example, the same instrument can be used to prepare a tibial plateau regardless of whether the tibia island is being retained or resected. Any suitable instrumentation can be used, such as the tibial cut guide and vertical wall guide of the Vanguard XP® instrument system by Biomet, Inc. Any suitable common instrumentation can also be used to perform posterior resection for the femoral condyles in preparation for implanting either the unicondylar femoral component 510 or the total femoral component 610.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used in this application is for the purpose of describing particular example embodiments only and is not intended to be limiting. The singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). The term “and/or” includes any and all combinations of one or more of the associated listed items.

Although the terms first, second, third, etc. may be used to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An arthroplasty system comprising:
a unicondylar tibial tray implant;
a total tibial tray implant; and
a tibial bearing configured to individually couple with each one of the unicondylar tibial tray implant and the total tibial tray implant.

2. The arthroplasty system of claim 1, wherein the unicondylar tibial tray implant includes a unicondylar bearing engaging surface and is one of a medial unicondylar tibial tray implant or a lateral unicondylar tibial tray implant;
wherein when the unicondylar tibial tray implant is a medial unicondylar tibial tray implant, the unicondylar bearing engaging surface is substantially similar to a medial bearing engaging surface of the total tibial tray implant in size and shape; and
wherein when the unicondylar tibial tray implant is a lateral unicondylar tibial implant, the unicondylar bearing engaging surface is substantially similar to a lateral bearing engaging surface of the total tibial tray implant in size and shape.

3. The arthroplasty system of claim 1, wherein the unicondylar tibial tray implant includes a unicondylar tibial tray implant coupling system and the total tibial tray implant includes a total tibial tray implant coupling system that is substantially similar to the unicondylar tibial tray implant coupling system.

4. The arthroplasty system of claim 1, further comprising a plurality of each one of the unicondylar tibial tray implant, the total tibial tray implant, and the tibial bearing all sized and shaped at uniform intervals based on the same anatomic data.

5. The arthroplasty system of claim 1, wherein the total tibial tray implant defines an intercondylar box.

6. The arthroplasty system of claim 1, further comprising a total tibial bearing configured to cooperate with the total tibial tray implant, the total tibial bearing including a total medial bearing and a total lateral bearing;
wherein the total medial bearing is substantially similar to the tibial bearing in size and shape when the tibial bearing is medial; and
wherein the total lateral bearing is substantially similar to the lateral bearing in size and shape when the tibial bearing is lateral.

7. The arthroplasty system of claim 1, wherein the tibial bearing includes a plurality of tibial bearings each configured to provide different levels of constraint, each one of the plurality of tibial bearings configured to couple with the unicondylar tibial tray implant and the total tibial tray implant.

8. The arthroplasty system of claim 1, further comprising a unicondylar femoral implant and a total femoral implant, each of which is configured to individually articulate with the tibial bearing.

9. The arthroplasty system of claim 8, wherein the unicondylar femoral implant includes a unicondylar femoral articulating surface, and the total femoral implant includes both a first total femoral articulating surface and a second total femoral articulating surface; and
wherein the unicondylar femoral articulating surface is substantially similar to at least one of the first or the second total femoral articulating surfaces in size and shape.

10. An arthroplasty system comprising:
a unicondylar tibial tray implant including a unicondylar bearing engaging surface; and
a total tibial tray implant including a medial bearing engaging surface and a lateral bearing engaging surface;
wherein the unicondylar bearing engaging surface is substantially similar to the medial or the lateral bearing engaging surfaces in size and shape.

11. The arthroplasty system of claim 10, wherein the unicondylar bearing engaging surface is identical to the medial or the lateral bearing engaging surface in size and shape.

12. The arthroplasty system of claim 10, wherein when the unicondylar tibial tray implant is a medial unicondylar tibial tray implant, the unicondylar bearing engaging surface is substantially similar to the medial bearing engaging surface in size and shape; and
wherein when the unicondylar tibial tray implant is a lateral unicondylar tibial tray implant, the unicondylar bearing engaging surface is substantially similar to the lateral bearing engaging surface in size and shape.

13. The arthroplasty system of claim 10, wherein the unicondylar bearing engaging surface defines an outer unicondylar curve radius, the medial bearing engaging surface defines an outer medial curve radius, and the lateral bearing engaging surface defines an outer lateral curve radius;
wherein when the unicondylar bearing engaging surface is a lateral unicondylar bearing engaging surface, the unicondylar curve radius is the same as the outer lateral curve radius; and
wherein when the unicondylar bearing engaging surface is a medial unicondylar bearing engaging surface, the unicondylar curve radius is the same as the outer medial curve radius.

14. The arthroplasty system of claim 10, further comprising:
a tibial bearing configured to individually couple with the unicondylar tibial tray implant at the unicondylar bearing engaging surface and the total tibial tray implant at one of the medial bearing engaging surface or the lateral bearing engaging surface;
a unicondylar femoral implant; and
a total femoral implant;
wherein each one of the unicondylar femoral implant and the total femoral implant are configured to articulate with the tibial bearing; and
wherein each one of the tibial bearing, the unicondylar femoral implant, the total femoral implant, the unicondylar tibial tray, and the total tibial tray are based on the same anatomic data.

15. An arthroplasty system comprising:
a unicondylar tibial tray implant including a unicondylar bearing engaging surface;
a total tibial tray implant including a medial bearing engaging surface, a lateral bearing engaging surface, and an intercondylar portion between the medial bearing engaging surface and the lateral bearing engaging surface, the unicondylar bearing engaging surface is substantially similar to the medial or the lateral bearing engaging surfaces in size and shape;
a tibial bearing configured to couple with the unicondylar tibial tray implant at the unicondylar bearing engaging surface, and configured to couple with the total tibial tray implant at one of the medial bearing engaging surface and the lateral bearing engaging surface;
a unicondylar femoral implant; and
a total femoral implant;
wherein each one of the unicondylar femoral implant and the total femoral implant are configured to articulate with the tibial bearing; and
wherein each one of the unicondylar tibial try implant, the total tibial tray implant, the tibial bearing, the unicondylar femoral implant, and the total femoral implant are sized and shaped based on the same anatomic data.

16. The arthroplasty system of claim 15, wherein the unicondylar tibial tray implant includes a unicondylar tibial tray implant bearing coupling system and the total tibial tray implant includes a total tibial tray implant bearing coupling system that is substantially similar to the unicondylar tibial tray implant bearing coupling system.

17. The arthroplasty system of claim 15, further comprising a total tibial bearing configured to couple with the total tibial tray implant, the total tibial bearing including a total medial bearing portion, a total lateral bearing portion, and a post between the total medial bearing portion and the total lateral bearing portion, the total medial bearing portion is substantially similar to the tibial bearing in size and shape when the tibial bearing is medial, and the total lateral bearing portion is substantially similar to the lateral bearing portion in size and shape when the tibial bearing is lateral.

18. The arthroplasty system of claim 15, wherein the tibial bearing is one of a plurality of tibial bearings each configured to provide different levels of constraint, each one of the plurality of tibial bearings configured to independently couple with the unicondylar tibial tray implant and the total tibial tray implant.

19. The arthroplasty system of claim 15, further comprising a plurality of each one of the unicondylar tibial tray implant, the total tibial tray implant, the tibial bearing, the unicondylar femoral implant, and the total femoral implant all sized and shaped at uniform intervals based on the same anatomic data.

20. The arthroplasty system of claim 15, wherein the tibial bearing is configured to couple with a plurality of different coupling systems in order to couple the tibial bearing to the unicondylar tibial tray implant and the total tibial tray implant.
